(19) United States
(12) Patent Application Publication
(10) Pub. No.: US 2009/0012494 A1
(43) Pub. Date: Jan. 8, 2009

(54) INTRADERMAL DELIVERY OF BIOLOGICAL AGENTS

(75) Inventors: Yehoshua Yeshurun, Haifa (IL); Yotam Levin, Nes Ziona (IL); Yotam Almagor, Jerusalem (IL); Gilad Lavi, Rishon Letzion (IL); Meir Hefetz, Oshrat (IL); Yoel Sefi, Kibbutz Malkiya (IL); Richard Ian Catchpole, Hertfordshire (GB)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: NanoPass Technologies Ltd., Nes Ziona (IL)

(21) Appl. No.: 11/946,889

(22) Filed: Nov. 29, 2007

(30) Foreign Application Priority Data
Oct. 17, 2006 (GB) 0620617
Oct. 17, 2007 (IL) PCT/IL2007/001244

(51) Int. Cl. A61M 5/32; A61K 33/00; A61K 38/09; A61K 38/22; A61K 31/70; A61K 31/7105; A61K 31/485; A61K 38/47; A61K 38/16; A61K 38/28; A61K 38/21; A61K 38/20; A61K 31/4748; A61K 31/727; A61K 31/166; A61K 31/551; A61K 39/395; A61K 31/465; A61K 31/702; A61K 31/4178; A61K 31/137; A61K 39/12; A61K 39/00; A61K 39/02; A61K 39/235; A61K 39/21; A61K 39/155; A61K 39/285; A61K 39/145; A61K 39/245; A61K 39/295; A61P 43/00

(52) U.S. Cl. 604/506; 424/600; 514/2; 514/12; 514/23; 514/44; 514/289; 424/94.61; 514/8; 514/3; 424/85.4; 424/85.2; 514/305; 514/56; 514/619; 514/221; 424/141.1; 514/343; 514/54; 514/397; 514/646; 514/169; 424/204.1; 424/184.1; 424/234.1; 424/233.1; 424/207.1; 424/218.1; 424/211.1; 424/232.1; 424/209.1; 424/231.1; 424/202.1

(57) ABSTRACT

The present invention relates to methods for intradermally delivering one or more biologically active agents such as vaccines and therapeutic agents into the dermis layer of the skin of a subject to obtain systemic delivery or an immune response using a microneedle drug delivery device containing the agent to be delivered. The methods employ a microneedle device with a row of hollow microneedles. The microneedles penetrate the skin of the subject and assume an anchored state in which the microneedles are anchored in the skin and project laterally from the device. A pivotal motion is then performed with the device so that the skin in which the microneedles are engaged is lifted above the initial plane of the surface of the skin while the biologically active agent is delivered. The methods of the invention elicit increased humoral and/or cellular response as compared to conventional vaccine delivery routes, facilitating dose sparing.

This application is a continuation-in-part of International Application No. PCT/IL2007/001244 filed Oct. 17, 2007, which itself benefits from the priority of United Kingdom provisional patent application no. GB 0620617 filed Oct. 17, 2006, both of which are incorporated herein by reference in their entirety.

### FIELD AND BACKGROUND OF THE INVENTION

The present invention relates to methods for delivering biologically active agents such as vaccines, therapeutic and diagnostic agents into the intradermal layer of the skin of a subject using a microneedle device for drug delivery with geometry that allows a controllable, consistent and shallow delivery depth. The methods of the present invention elicit increased humoral and/or cellular response as compared to conventional vaccine delivery methods, e.g., intramuscular (NM) or subcutaneous (SC) route. Furthermore, the methods of the present invention facilitate induction of a comparable immune response by an amount of vaccine that is significantly lower than the currently used full dose when delivered via conventional vaccine routes, e.g., intramuscular route.

Approaches for injecting substances beneath the surface of the skin have almost exclusively involved subcutaneous, intramuscular or intravenous routes of administration of which intramuscular (IM) and subcutaneous (SC) injections have been the most commonly used. The skin is the largest and the most accessible organ on the human body. It offers a protective barrier of only a couple of millimeters between the external environment and the blood circulation. An individual's age, sex, and race, as well as the pathophysiology and anatomical location of the skin, influence the thickness (1) and permeability of the skin to drugs. The skin is a very effective barrier against many drugs, in particular those with high molecular weights. In addition to serving as a mechanical and chemical barrier, the skin serves as an important immune system organ. Antigen presenting cells (APC), specifically Langerhans cells that reside mostly in the epidermis, as well as dermal dendritic cells (which reside mostly in the dermis), are activated by the presence of immune stimulants (antigens from viral, bacterial, or parasitic origin), take in the antigen (via phagocytosis and/or endocytosis) and migrate to the regional draining lymph nodes, where presentation to the immune system occurs, eliciting a robust immune response. Those characteristics serve as the basis for biological benefits of intradermal (ID) drug delivery.

Dermal tissue represents an attractive target site for delivery of vaccines and gene therapeutic agents. Several gene therapeutic agents are designed for the treatment of skin disorders, skin diseases and skin cancer. In such cases, direct delivery of the therapeutic agent to the affected skin tissue is desirable. In addition, skin cells are an attractive target for gene therapeutic agents, of which the encoded protein or proteins are active at sites distant from the skin. In such cases, skin cells (e.g., keratinocytes) can function as “bioreactors” producing a therapeutic protein that can be rapidly absorbed into the systemic circulation via the papillary dermis. In other cases, direct access of the vaccine or therapeutic agent to the systemic circulation is desirable for the treatment of disorders distant from the skin. In such cases, systemic distribution can be accomplished through the papillary dermis. Further, there are select diagnostic applications (e.g., the injection of Tuberculin, or PPD, for the diagnosis of Tuberculosis exposure (2), Chagas disease and Leishmaniasis, that are directed at the skin and are delivered intradermally. However, intradermal (ID) injection using standard needles and syringes is technically very difficult to perform and is painful. According to some publications (3), the leakage rate of application using conventional needles has been measured at 19-41% (medium and large leakage included). Moreover, it is estimated that in many cases the substance is delivered too deep, into the SQ space, and produces a lesser immunogenic/diagnostic effect. The prior art contains several references to ID delivery of both DNA-based and conventional vaccines and therapeutic agents, however results have been conflicting, at least in part due to difficulties in accurately targeting the ID tissue with existing techniques. Microneedles represent a novel drug delivery method that offers a convenient and efficacious ID drug delivery. Microneedles are produced by adapting the tools of micro-electro-mechanical (MEMS) technology. The microneedles penetrate hundreds of microns into the skin in a painless manner and enable accurate and reliable intradermal delivery of the drug. Intradermal (ID) drug delivery may offer several biological benefits for patients, both in terms of vaccine and non-vaccine applications.

### Vaccine Applications

Studies older (4, 5) and recent (6, 7), demonstrate that intradermal vaccination with various vaccines require as little as 10-40% the dose of standard intramuscular (IM vaccination) for a similar or improved response. This is true not only for vaccines that may be given intradermally as a primary route (BCG, Yellow Fever and Small Pox) but also for others that are usually given SC or IM (Influenza and Hepatitis B) (8). Animal studies demonstrate that ID delivery of various antigens induce both cellular and humoral responses. This may offer an improved immunogenicity. Further, enhanced immunogenicity can reduce the number of boosts required for an effective vaccination, potentially also increasing longevity of protection.

Based on an improved immunogenicity, ID delivery may allow higher titers of antibodies to be produced in animals too. This may allow improved antibody production for diagnostics and research, for example, in the production of analysis kits. The higher antibody production yield can present itself in either higher titers for the same period of time, reduction of boosts required for the target titer, faster arrival to target titers or a combination thereof. Antibodies could be used for multiple applications presented in the literature (including antibodies for human therapeutics).

A wide variety of substances can be delivered intradermally, such as but not limited to, a vaccine or a medicament comprising genetic material to afford an immunogenic or therapeutic response, live/non-live attenuated virus/bacteria with/without adjuvant, nucleic acids, peptides, proteins, carbohydrates, polysaccharides and whole cells, lipids, organic molecules, biologically active inorganic molecules, and combinations thereof used in the prevention, diagnosis, alleviation, treatment, or cure of diseases.

Using microneedles for ID vaccination is more reliable, has a more consistent delivery depth (due to the depth limitation exercised by the substrate microneedle array), is easier to perform (in comparison to standard hypodermic needles) and thus, provides a good solution for clinical practice. Potentially, the use of microneedles may improve the benefits not only compared with IM and SC injection but over conventional intradermal delivery using regular needles. Additionally, using microneedles for ID vaccination is less painful and less intimidating for patients, thus improving compliance, especially for target populations such as children.

### Non-Vaccine Applications

Microneedle delivery may offer additional benefits for non-vaccine applications. For example, ID delivery using microneedles may improve the pharmacokinetic profile: shorter T max, higher C max, higher bioavailability, more rapid uptake rates, more rapid onset of pharmacodynamics or biological effects, reduced drug depot effects, decreased lag time in general, and reduction of undesired immune responses (including for example immunomodulation).

Non-vaccine applications which may provide such benefits when delivered by microneedles include, but are not limited to: anesthetics, peptides and proteins including Insulin, allergy test antigens, diagnostics including but not limited to the Mantoux and other skin tests, cytokines, chemokines, hormones, immunomodulatory and therapeutic proteins including but not limited to monoclonal antibodies and fusion proteins comprised of antibodies, antigen-binding domains of antibodies or other fragments thereof and dermal fillers or aesthetic agents such as but not limited to HA, collagen and Botox.Bolus ID substance administration results in kinetics more similar to IV injection. ID injections are closer to the capillary bed, therefore they may allow improved and more rapid absorption and systemic distribution, in comparison to the SC and the IM routes. ID delivery using microneedles may improve the delivery of a medicament (chemical, biological, synthetic or genetic material) for the treatment of skin diseases, genetic skin disorders or skin cancer by accurately targeting the dermis, as well as for diagnostics.

The most common injection sites for vaccines are the upper arm (over the deltoids), and buttocks. Insulin and EPO are usually injected in the lower outer quadrants of the abdomen and on the upper thighs. Most vaccines are administered intramuscularly (IM) using conventional needles (sometimes attached to pre-filled syringes), while rh-EPO and insulin are administered subcutaneously using automatic and pen injectors, pre-filled syringes, infusion sets or standard syringes.

Intradermal injections typically required significant expertise on behalf of the user. Even in experienced hands success rates in intradermal injections are less than optimal (19-41% leakage in some publications (3)). This may result in reduced efficacy of the injected substance due to the lower than expected dose injected into the appropriate space. Accordingly, there is a long-felt need for a simplified method of intradermally delivering one or more biological agents which overcomes the problems and limitations associated with conventional methods, by making such injections less dependent upon experience and technique. In addition, there is a need to limit the depth of penetration of the microneedle arrangement into the skin of the subject to avoid entry into the subcutaneous layer of the skin as well as reliably fixing the orientation of the microneedle relative to the skin, and to achieve consistent results between users, between injections of the same user, between different skin types, subject's age and the like.

### Microneedle Devices

Of particular relevance as background to the present invention are PCT patent application publication no. WO 2005/049107 A2 and US patent application publication no. 2005/0209566, both commonly assigned with the present invention, which are hereby incorporated by reference in their entirety. These documents disclose a system and method for delivering fluid into a flexible biological barrier employing a microneedle structure wherein a final position of microneedles inserted into the biological barrier is generally sideways projecting from the delivery configuration instead of the conventional downwards projecting arrangement. This technique is referred to herein for convenience as “side insertion.” The microneedles project from a relief surface which is distinct from a primary biological-barrier contact region of the delivery configuration, and is typically angled upwards so that it is not in face-on relation to the biological barrier. During insertion, the contact region is brought into contact with the biological barrier and moved substantially parallel to the surface of the flexible biological barrier so as to generate a boundary between a stretched portion and a non-stretched portion of the barrier. Typically concurrently with this motion, the microneedles penetrate into the flexible biological barrier such that, at the end of the motion, the microneedles extend into the flexible biological barrier from the boundary region in a direction towards the non-stretched portion. Fluid is then injected through the bores of the hollow microneedles towards the non-stretched portion.

Preferred microneedle designs for implementing the aforementioned device are structures similar to those disclosed in U.S. Pat. No. 6,533,949, also co-assigned with the present invention, which is hereby incorporated by reference in its entirety. The needles described therein have a generally triangular cross-sectional shape including one or more upright wall intersecting with a sloped surface (referred to below as the “bevel surface” of the needle) through which a fluid flow channel passes.

A major factor that has precluded the widespread use of the ID delivery route and has contributed to the conflicting results described above is the lack of suitable devices to accomplish reproducible delivery to the epidermal and dermal skin layers. Standard needles commonly used to inject vaccines are too large to accurately target these tissue layers when inserted into the skin. The most common method of delivery is through Mantoux-style injection using a standard needle and syringe. This technique is difficult to perform, unreliable and painful to the subject. Thus, there is a need for devices and methods that will enable efficient, accurate and reproducible delivery of drugs, vaccines, diagnostic and therapeutic agents to the intradermal layer of skin, and that would provide reliable shallow drug delivery while minimizing deformation of the skin, substantially without pain to the patient.

### SUMMARY OF THE INVENTION

In contrast to the conventional methods discussed above, the present invention is directed to improved, more efficient methods for intradermally delivering at least one biologically active agent into the skin of a subject to obtain systemic delivery or an immune response, using a microneedle device. The agent may comprise drugs, vaccines or a medicament comprising genetic material, live/non-live attenuated virus/bacteria with/without adjuvant; nucleic acids, peptides, proteins, carbohydrates, polysaccharides, whole cells, lipids, organic molecules, biologically active inorganic molecules, and combinations thereof. Compared with the standard routes (intravenous, intramuscular, subcutaneous) of drug insertion, the methods of the invention offers significant biological benefits for the patients in terms of safety (as the device cannot penetrate deeper than the dermis) with minimal expertise in intradermal injections required. The methods of the invention improves the efficacy of vaccinations, results in improved immunogenicity due to the rich distribution of immune cells in the dermis, reduces the required effective dose (due to elimination of first pass effect), improves absorption rates (due to the rich venous plexus in the dermis), improves the ease of delivery and reduces pain. It is also possible to self-administer intradermal injections by the methods of the invention.

The microneedle injection technique proposed in this invention allow excellent control over delivery depth, and allows particularly shadow delivery. Injection to shallow depths of skin typically offers one or more of the following advantages:
1. The shallow layers of skin contain no pain sensing nerves, and thus injections preformed with the current invention cause minimal pain or no pain at all.
2. The shallow layers of skin contain a rich network of blood vessels which enable rapid absorption of injected material.
3. Use of microneedle devices also eliminates the chances of unintended injection into subcutaneous tissue, muscle, or blood vessels, all of which are located deeper than the maximal penetration depth of the microneedle devices.

According to the teachings of the present invention there is provided, a method of intradermally delivering at least one biologically active agent into the skin of a subject, comprising the steps of: (a) providing a microneedle device including at least one hollow microneedle; (b) causing the at least one microneedle to penetrate into the skin of the subject and assume an anchored state in which the microneedle is anchored in the skin and projects from the microneedle device in a direction having a major component parallel to the initial plane of the surface of the skin; (c) performing a pivotal motion of the microneedle device such that the portion of the skin in which the at least one microneedle is engaged is lifted above the initial plane of the surface of the skin; and (d) while the portion of the skin is lifted, delivering intradermally via the microneedle a quantity of a biologically active agent.

According to a further feature of the present invention, the pivotal motion is performed such that a region of the device behind the microneedle is pressed against the skin, thereby enhancing sealing against the skin.

According to a further feature of the present invention, the pivotal motion is performed by angular motion through an angle of between about 5 degrees and about 30 degrees.

According to a further feature of the present invention, the microneedle device is associated with a syringe, and wherein the pivotal motion is performed by pressing the syringe substantially flat against the surface of the skin.

According to a further feature of the present invention, the microneedle device comprises: (a) a skin contact surface which is brought into substantially facing relation with the skin surface in the anchored state; (b) a front surface angled relative to the skin contact surface and meeting the skin contact surface at an edge, the at least one hollow microneedle being deployed so as to project from the front surface adjacent to the edge; and (c) a rear surface angled relative to the skin contact surface and meeting the skin contact surface at a pivot region about which the pivotal motion is performed.

According to a further feature of the present invention, the at least one hollow microneedle is implemented as a plurality of hollow microneedles deployed in a line adjacent to the edge.

According to a further feature of the present invention, the at least one microneedle is formed on a substrate, the microneedle projecting from the substrate to a height of between about 250 and about 750 microns.

According to a further feature of the present invention, the agent is selected from the group consisting of drugs, vaccines, peptides, proteins, carbohydrates, nucleic acid molecules, lipids, organic molecules, biologically active inorganic molecules, and combinations thereof used in the prevention, diagnosis, alleviation, treatment, or cure of diseases.

According to a further feature of the present invention, the nucleic acid molecules include DNA, cDNA, RNA, siRNA, oligonucleotides, genes and a fragment thereof.

According to a further feature of the present invention, the drugs include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as hirulog, IgE suppressors, Insulin, insulinotropin and analogs, Insulin-like growth factors, Interferons, Interleukins, Leutenizing hormone, Leutenizing hormone releasing hormone and analogs, Low molecular weight heparin, M-CSF, metoclopramide, Midazolam, Monoclonal antibodies, Narcotic analgesics, nicotine, Non-steroid anti-inflammatory agents, Oligosaccharides, ondansetron, Parathyrold hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, the vaccines, with or without carriers/adjuvants, include prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimers, H. Pylori, salmonella, diabetes, cancer, herpes simplex, human papilloma and like other agents include all of the major therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, anti-depressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, sexual hypofunction and tranquilizers and major diagnostics such as tuberculin and other hypersensitivity and allergy agents.

According to a further feature of the present invention, the agent is a vaccine, with or without carriers, adjuvants and vehicles, the vaccine selected from the group consisting of prophylactic and therapeutic antigens including but not limited to subunit proteins, peptides, polypeptides, and polysaccharides, polysaccharide conjugates, toxoids, genetic based vaccines, live attenuated bacteria or viruses, mutated bacteria or viruses, reassortant bacteria or viruses; live, non-attenuated virus or viral vectors, inactivated or killed virus, live, non-attenuated bacteria and inactivated or killed bacteria or viruses, whole cells or components thereof, cellular vaccines or components thereof, viral and bacterial vectors including but not limited to those derived from adenoviruses, retroviruses alphaviruses, flaviviruses, and vaccinia viruses, vaccines in connection with addiction, anthrax, arthritis, cholera, diphtheria, dengue, tetanus, lupus, multiple sclerosis, parasitic diseases including Leishmania, psoriasis, Lyme disease, SARS, Ebola, Yellow Fever, meningococcus, measles, mumps, rubella, varicella, yellow fever, Respiratory syncytial virus, tick borne Japanese encephalitis, pneumococcus, smallpox, streptococcus, staphylococcus, typhoid, influenza including seasonal, pre-pandemic and pandemic influenza, hepatitis, including hepatitis A, B, C, D, E and G, otitis media, rabies, polio, HIV, parainfluenza, rotavirus, Epstein Barr Virus, CMV, chlamydia, non-typeable haemophilus, haemophilus influenza B (HIB), moraxella catarrhalis, human papilloma virus, tuberculosis including BCG, gonorrhoeae, asthma, atherosclerosis, malaria, E. coli, Alzheimer's Disease, H. Pylori, salmonella, diabetes, cancer, herpes simplex, human papilloma, Yersitiia pestis, traveler's diseases, West Nile encephalitis, Carnplobacter, and C. difficile and bioterrorism agents.

According to a further feature of the present invention, the agent is selected from a combination of vaccines against (i) measles, mumps and rubella, (ii) diphtheria, tetanus and acellular pertussis, (iii) hepatitis A and hepatitis B (iv) haemophilus influenza B, diphtheria, tetanus and acellular pertussis, (v) haemophilus influenza B, hepatitis B, diphtheria, tetanus and a cellular pertussis, and (vi) haemophilus influenza B, inactivated polio, diphtheria, tetanus and acellular pertussis which are commonly delivered in combination to the subcutaneous and/or intramuscular space.

According to a further feature of the present invention, the agent is at least one vaccine and wherein a comparable immune response is induced using a lesser dose of the agent as compared to when the same agent is delivered via an intramuscular route.

According to a further feature of the present invention, the agent achieves a better response using the same dose, or an equivalent response using a lesser dose, lesser adjuvant or no adjuvant, faster onset, longer response, improved memory response, higher bioavailability, lesser boosts, and lesser toxicity compared to when the same agent is delivered via an intramuscular route.

According to a further feature of the present invention, the agent achieves a similar response in less time or a greater response in similar time as compared to when the same agent is delivered via an intramuscular route.

According to a further feature of the present invention, the agent achieves a greater antibody production yield by generating higher titers for the same period of time or reduction of boosts required for the target titer or faster arrival to target titers or a combination thereof as compared to when the same agent is delivered via an intramuscular or subcutaneous route.

According to a further feature of the present invention, the agent is delivered at a controlled rate, volume, pressure and depth to generate a Mantoux skin response more reliably and consistently than the traditional intradermal Mantoux method with reduced false negative skin diagnostics.

### BRIEF DESCRIPTION OF THE DRAWINGS

The invention is herein described, by way of example only, with reference to the accompanying drawings, wherein:

FIG. 1A is a schematic isometric view of a microneedle device employed for implementing the intradermal delivery method according to the teachings of the present;

FIG. 1B is a cross-sectional view taken through the microneedle device of FIG. 1A;

FIG. 2 is a sequence of schematic views illustrating the method of intradermal delivery according to the teachings of the present invention applied to the shoulder of a subject;

FIG. 3 is a sequence of schematic side views further illustrating the method of intradermal delivery according to the teachings of the present invention applied to a region of skin;

FIGS. 4A and 4B are enlarged schematic cross-sectional views showing the interface of the microneedle device of FIG. 1B with the surface of the skin of a subject at the end of an anchoring motion and after a subsequent pivotal motion, respectively;

FIG. 5A is a bar graph comparing the levels of antigen/antibody complex levels detected by radioimmunoassay following immunization with 25 g hypodermic needle (“regular”) and microneedle (“Nano”);

FIG. 5B is a bar graph comparing the levels of antiserum titer detected by radioimmunoassay following immunization with 25 g hypodermic needle (“regular”) and microneedle (“Nano”);

FIG. 6A-FIG. 6C are bar graphs comparing the geometric mean titer (GMT) for strains H1NI (FIG. 6A), H3N2 (FIG. 6B) and B (FIG. 6C) for intradermal low dose (ID1), intradermal medium dose (ID2) and intramuscular (IM/standard) vaccination;

FIG. 7A-FIG. 7C are bar graphs comparing the seroconversion factor for strains H1NI (FIG. 6A), H3N2 (FIG. 6B) and B (FIG. 6C) for intradermal low dose (ID1), intradermal medium dose (ID2) and intramuscular (N/standard) vaccination;

FIG. 8A-FIG. 8C are bar graphs comparing the seroconversion rate for strains H1NI (FIG. 6A), H3N2 (FIG. 6B) and B (FIG. 6C) for intradermal low dose (ID1), intradermal medium dose (ID2) and intramuscular (IM/standard) vaccination; and

FIG. 9A-FIG. 9C are bar graphs comparing the seroprotection rate for strains H1NI (FIG. 6A), H3N2 (FIG. 6B) and B (FIG. 6C) for intradermal low dose (ID1), intradermal medium dose (ID2) and intramuscular (IM/standard) vaccination.

### DESCRIPTION OF THE PREFERRED EMBODIMENTS

The present invention is method of intradermally delivering at least one biologically active agent into the skin of a subject.

The principles and operation of methods according to the present invention may be better understood with reference to the drawings and the accompanying description.

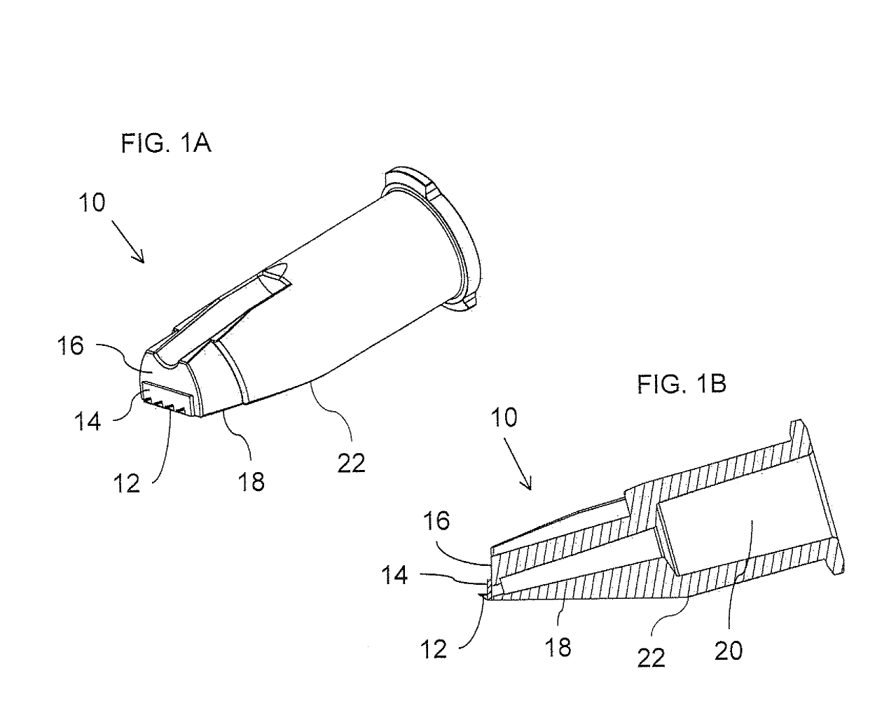

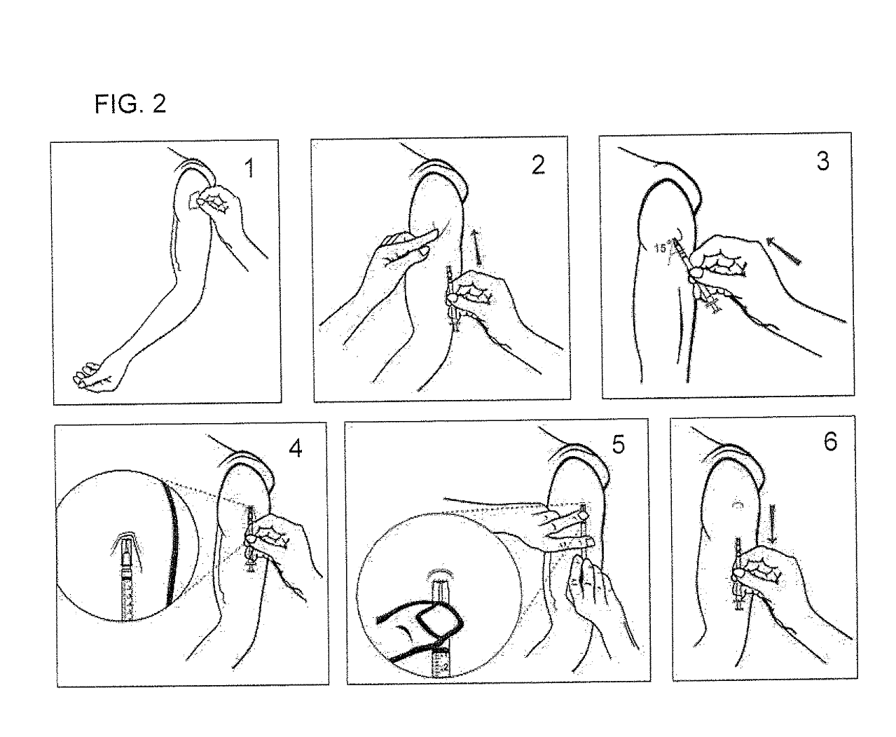

Referring now to the drawings, FIGS. 1A and 1B illustrate an example of a microneedle drug delivery device for delivery of fluids to intradermal layers of the skin of a mammalian subject that can be used to practice the methods of the present invention. The principles and operation of devices according to the present invention may be better understood with reference to the drawings and the accompanying description.

The method of the present invention can be used to intradermally deliver agents such as drugs, vaccines, peptides, proteins, carbohydrates, nucleic acid molecules, lipids, organic molecules, biologically active inorganic molecules, and combinations thereof used in the prevention, diagnosis, alleviation, treatment, or cure of disease into the skin of a subject such as a human.

Referring now to the drawings, FIGS. 1A and 1B show an implementation of a microneedle device, generally designated 10, constructed and operative according to the teachings of the present invention, illustrating the flow channel aspect of the present invention. The microneedle-skin interface of device 10 includes a linear array of microneedles 12 projecting from a substrate 14 attached to a relief surface 16 adjacent to a skin contact surface 18, and where the relief surface and skin contact surface are roughly orthogonal. A primary flow channel 20 is in fluid connection to bores passing through the microneedles and extends rearwards through the device for connecting to a syringe. In the case shown here, the rear portion of flow channel 20 is formed with a slightly conical diverging shape configured to provide a standard female luer connector for engagement on a syringe tip. It should be noted, however, that the invention is not limited to such an implementation, and may instead be implemented, for example, as an integral part of a prefilled or disposable syringe.

It will be noted that flow channel 20, defining the orientation of attachment of the syringe, has an axial direction inclined at an acute angle relative to skin contact surface 18, in this case of around 15 degrees. This angle, or the corresponding geometrical relation between the syringe axis and the skin contact surface in integrally formed embodiments, plays a significant role in defining the range of a pivotal motion performed as part of the injection method of the present invention, as will be detailed further below. Another parameter of significance is the extent of the skin contact surface extending rearwards from microneedles 12. In the case illustrated here, microneedle device 10 is formed primarily from a molded block which provides the various external surfaces and internal channels described herein. The adapter is preferably implemented in a substantially cylindrical form with which the skin contact surface 18 intersects at a pivot region 22. The pivot region 22 is preferably between about 0.5 and about 1.5 centimeters from the microneedles 12, and most preferably no more than 1 centimeter. In alternative implementations, it should be noted that the pivotal motion to be described below may employ pivotal contact at a location separate from contact surface 18. In this case, contact surface 18 may be made significantly shorter, and is essentially without a minimum limit to its length.

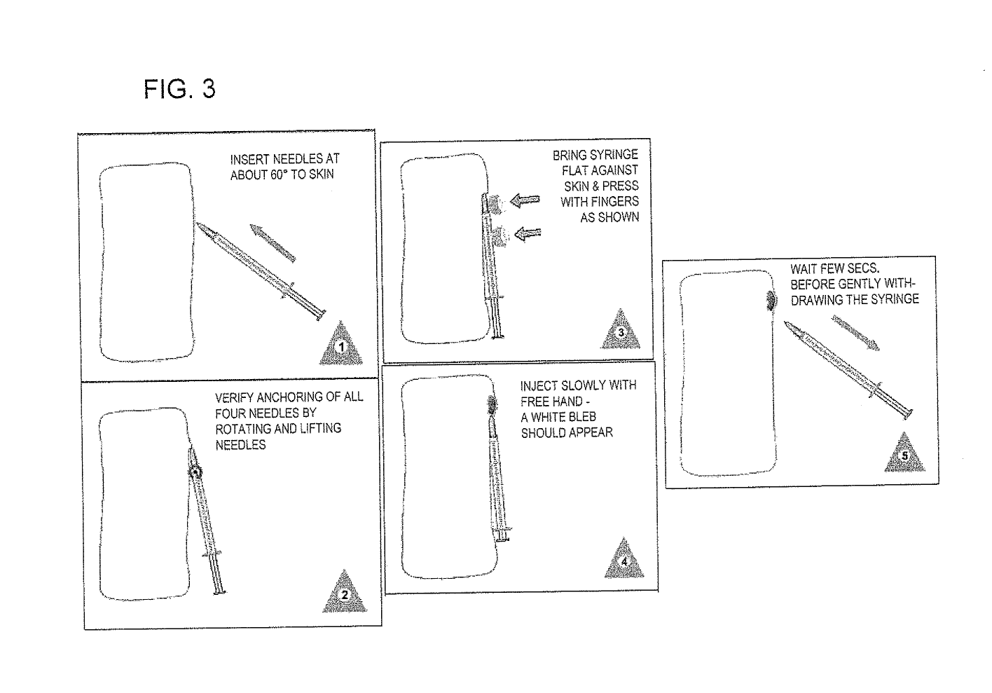

Turning now to the injection method of the present invention itself, this is will be described below in more detail with reference to FIGS. 2-4B. The invention provides a method for intradermally delivering at least one biologically active agent into the skin of a subject. In general terms, the method employs a microneedle device including a number of hollow microneedles, such as device 10 described above. The user first causes the microneedles to penetrate into the skin of the subject and assume an anchored state in which the microneedle is anchored in the skin and projects from the microneedle device in a direction having a major component parallel to the initial plane of the surface of the skin. A pivotal motion of the microneedle device is then performed such that the portion of the skin in which the microneedles are engaged is lifted above the initial plane of the surface of the skin. Wile the portion of the skin is so lifted, a quantity of a biologically active agent is delivered intradermally via the microneedles. This lifting action is believed to contribute to the unusually shallow intradermal delivery of the biologically active agent, thereby achieving various advantages which will be discussed further below. Further details of the method and its various advantages will be more fully described below.

Before addressing the features of various specific implementations of the present invention in more detail, it will be useful to define certain terminology as used herein in the description and claims. Firstly, the device is described as delivering a fluid into a flexible biological barrier. While the invention may be used to advantage for delivery of fluids (as defined below) through a wide range of biological barriers including the skin, the walls of various internal organs or vessels, the oral mucosa barrier, gums and buccal surfaces, the blood-brain barrier, etc., the invention is primarily intended for delivery of fluids into layers of the skin of a mammalian subject, and in particular, for intradermal or intra-epidermal delivery of fluids into the skin of a human subject. It will be understood that any biological barrier can be substituted for “skin” throughout. The fluids delivered may be any fluids including gels, hydrogels, etc. Preferred examples include, but are not limited to, dermatological treatments, vaccines, and other fluids used for cosmetic, therapeutic or diagnostic purposes. Furthermore, although considered of particular importance for intradermal fluid delivery, it should be noted that the present invention may also be applied to advantage in the context of transdermal fluid delivery and/or fluid aspiration such as for diagnostic sampling. The invention encompasses intradermal vaccine delivery to treat and/or prevent an infectious disease in a subject, preferably a human. Infectious diseases that can be treated or prevented by the methods of the present invention are caused by infectious agents including, but not limited to, viruses, bacteria, fungi, protozoa, prions and parasites. As used herein, “intradermal” (ID) is intended to mean administration of a substance into the dermis in such a manner that the substance readily reaches the richly vascularized papillary dermis where it can be rapidly systemically absorbed, or in the case of vaccines (conventional and genetic) or gene therapeutic agents may be taken up directly by cells in the skin. The term “intradermal” is used generally herein to include every layer of the skin, including stratum corneum, epidermis and dermis. In the case of genetic vaccines, intended target cells include APC (including epidermal Langerhan's cells and dermal dendritic cells). In the case of gene therapeutic agents for diseases, genetic disorders or cancers affecting tissues distant from the skin, intended target cells include keratinocytes or other skin cells capable of expressing a therapeutic protein. In the case of gene therapeutic agents for diseases, genetic disorders or cancers affecting the skin, the intended target cells include those skin cells which may be affected by the disease, genetic disorder or cancer. As used herein, the term “biologically active agent” refers to a compound, molecule, or treatment that elicits a biological response from any host, animal, vertebrate, or invertebrate. Examples of active agents include therapeutic agents, diagnostics (compounds such as, but not limited to, dyes, gas, metals, or radioisotopes), pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, and an anti-tumor agent. The term “biologically active agent” further refers to the active agent, as well as its pharmacologically active salts, pharmaceutically acceptable salts, prodrugs, metabolites, analogs, and the like.

As used herein, the term “immunological response” or “immune response” to an antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells.

As used herein, and unless otherwise specified, the term “enhanced immune response” means that, when an antigenic or immunogenic agent of the invention is administered using the microneedle device of the invention, there is an increased antibody formation, measured using any standard methods known in the art, in a subject that receives such an administration as compared to a subject to which same amount of the antigenic or immunogenic agent is administered via IM, SC or other conventional route.

Alternatively, the term “enhanced immune response,” as used herein, means that, when an antigenic or immunogenic agent of the invention is administered using the microneedle device of the invention, a smaller amount of the antigenic or immunogenic agent can be used to achieve the same level of antibody formation in a subject, as compared to a subject to which the antigenic or immunogenic agent is administered via conventional routes. Methods for determining the efficacy of immunogenic compositions are known in the art and may be in vitro based assays or in vivo based assays, including animal based assays. Assays for measuring humoral immune response are well known in the art, e.g., see, Coligan et al., (eds.), 1997, Current Protocols in Immunology, John Wiley and Sons, Inc., Section 2.1. A humoral immune response may be detected and/or quantitated using standard methods known in the art including, but not limited to, an ELISA assay. The humoral immune response may be measured by detecting and/or quantitating the relative amount of an antibody which specifically recognizes an antigenic or immunogenic agent in the sera of a subject who has been treated with an immunogenic agent relative to the amount of the antibody in an untreated subject. ELISA assays can be used to determine total antibody titers in a sample obtained from a subject treated with an agent of the invention. In the case where the immunogenic agent comprises an influenza antigen any method known in the art for the detection and/or quantitation of an antibody response against an influenza antigen is encompassed within the methods of the invention.

As used herein, “ID Mantoux delivery” refers to the traditional ID Mantoux tuberculin test where an agent is injected at a shallow angle to the skin surface using a 27 or 30 gauge needle and standard syringe (3) (see, e.g., Flynn et al., which is incorporated herein by reference in its entirety). The Mantoux technique involves inserting the needle into the skin laterally, then “snaking” the needle further into the ID tissue. The technique is known to be quite difficult to perform and requires specialized training. A degree of imprecision in placement of the delivery results in a significant number of false negative test results. Moreover, the method involves a localized injection to elicit a response at the site of injection and the Mantoux approach has not led to the use of intradermal injection for systemic administration of agents. Reference is also made to geometrical relations to the surface of the flexible biological barrier. For the purpose of the present description and the appended claims, all geometrical relations to the “surface” of the flexible biological barrier are defined in relation to a plane approximating to the surface of the barrier in an initial state of rest of the biological barrier, i.e., prior to any deformation of the barrier caused by insertion of the microneedle fluid delivery configuration. As a more technical definition, particularly important in the case of a region of skin that has considerable curvature, this surface is defined as the plane containing two orthogonal tangents to the flexible biological barrier surface at the location of interest.

For convenience, directions or positions further from the surface of the skin are referred to as “up”, “above” or other similar terms, and directions or positions closer to, or deeper within, the skin are referred to as “down”, “below” or other similar terms. It will be understood that this terminology is arbitrary in the sense that the skin surface itself may have any orientation in space.

Where reference is made to a direction of motion having a component parallel to the surface of the biological barrier, this includes any motion that is not perpendicular to the skin surface. Preferably, the motion has a majority component parallel to the skin surface, i.e., at an angle shallower than 45 degrees. Most preferably, the part of the motion performed in contact with the skin is performed substantially parallel to the skin's surface, i.e., with a motion vector not more than about ±15 degrees above or below the plane of the skin surface at rest.

With regard to angles relative to the plane of the skin, angles will be referred to relative to a vector parallel to the skin as zero degrees with angles pointing into the skin being positive and angles away (outwards) from the skin being designated negative. For simplicity of presentation, use may be made of the term “upwards” or “up” to refer to directions outwards from the initial plane of the skin and “downwards” or “down” to refer to directions inwards or towards the initial plane of the skin.

Reference is also made to various physical states of the biological barrier. The biological barrier is described as “stretched” when a distance between points defined on the barrier in at least one direction is greater than the distance between the same two points when the skin is released. The direction of maximum strain is referred to simply as the stretching direction. “Unstretched” denotes a state of the skin where no stretching is present parallel to the direction of stretching in an adjacent region of stretched skin. It will be appreciated that, where compression of skin tissue has lead to local bulging or folding of the tissue, a degree of stretching may occur perpendicular to the compression vector to accommodate the out-of-plane distortion of the tissue. Nevertheless, such tissue is referred to herein as “unstretched” since no elongation is present in the direction of stretching. Tissue for which the distance between points is reduced relative to the same two points when the skin is released is referred to as “relaxed” tissue since it exhibits lower surface tension than the skin when released.

The present invention is referred to as employing one or more microneedles. The term “microneedle” is used herein in the description and claims to refer to a structure projecting from an underlying surface to a height of no more than 1 mm, and preferably having a height in the range of 250 to 750 microns. The microneedles employed by the present invention are preferably hollow microneedles having a fluid flow channel formed therethrough for delivery of fluid. The height of the microneedles is defined as the elevation of the microneedle tip measured perpendicularly from the plane of the underlying surface. The term “peripheral surface” is used to refer to any surface of the microneedle which is not parallel to the surrounding substrate surface. The term “upright” surface is used to refer to any surface which stands roughly perpendicular to the surrounding substrate surface.

As mentioned above, most preferred implementations of the present invention employ microneedles of a type similar to those disclosed in co-assigned U.S. Pat. No. 6,533,949, namely, formed with at least one wall standing substantially perpendicular to the underlying surface and deployed so as to define an open shape as viewed from above, the open shape having an included area, and an inclined surface inclined so as to intersect with the at least one wall, the intersection of the inclined surface with the at least one wall defining at least one cutting edge. The fluid flow channel is preferably implemented as a bore intersecting with the inclined surface. The particular robustness of the aforementioned microneedle structure and its particular geometrical properties exhibit great synergy with the structures and insertion methods of the present invention, ensuring that the microneedles can withstand the applied shear forces and are optimally oriented for delivery of fluids into the biological barrier. These advantages with be detailed further below.

Reference is also made to various surfaces which may be provided by a “block of material”. The term “block” is used herein to refer generically to any structure of one unitary element or plural elements cooperating to provide the recited surfaces in fixed mechanical relation. The “block” thus described includes, but is not limited to, a solid block, a hollow block, a thin sheet-like block and an open arrangement of surfaces mechanically interconnected to function together as a block. Part or all of the block may also be provided by a substrate upon which the microneedles are integrally formed. In certain preferred cases, a plurality of microneedles are deployed in a linear array, i.e., positioned spaced along a line.

The present invention relates to a “fluid transfer interface”, i.e., the structure and the operation of a microneedle arrangement which interfaces with the biological barrier to create a fluid transfer (delivery or sampling) path into or out through the barrier. The fluid transfer interface may be integrated as part of a self-contained fluid delivery device, or as an adapter device for use with an external fluid supply device. The term “fluid” is used to refer to any composition which flows, or can be induced to flow under working conditions of the device. Thus defined, “fluid” includes, but is not limited to, any and all types of liquid, gel, suspension or fluidized powder.

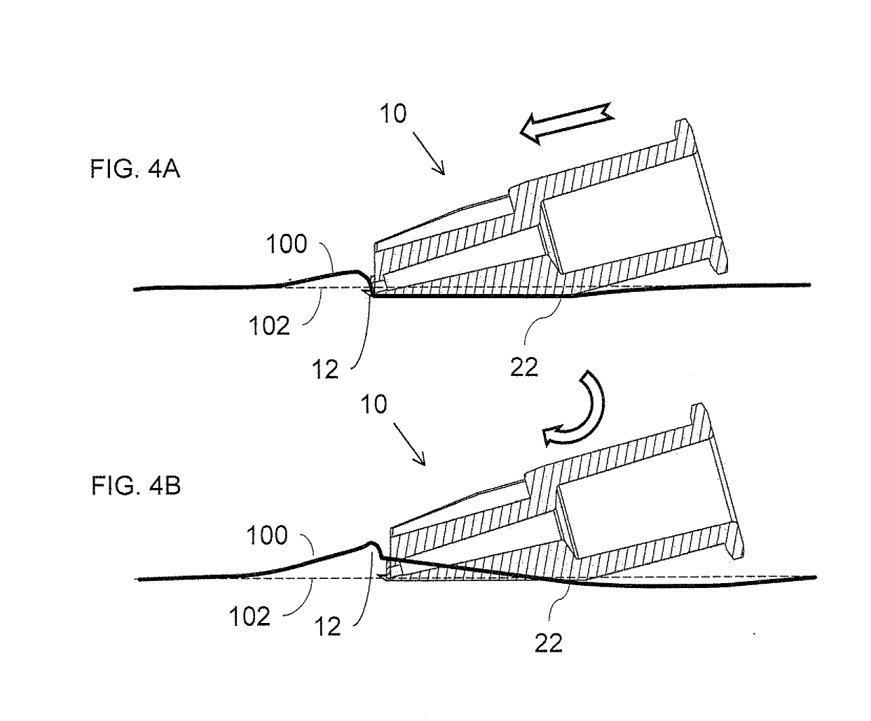

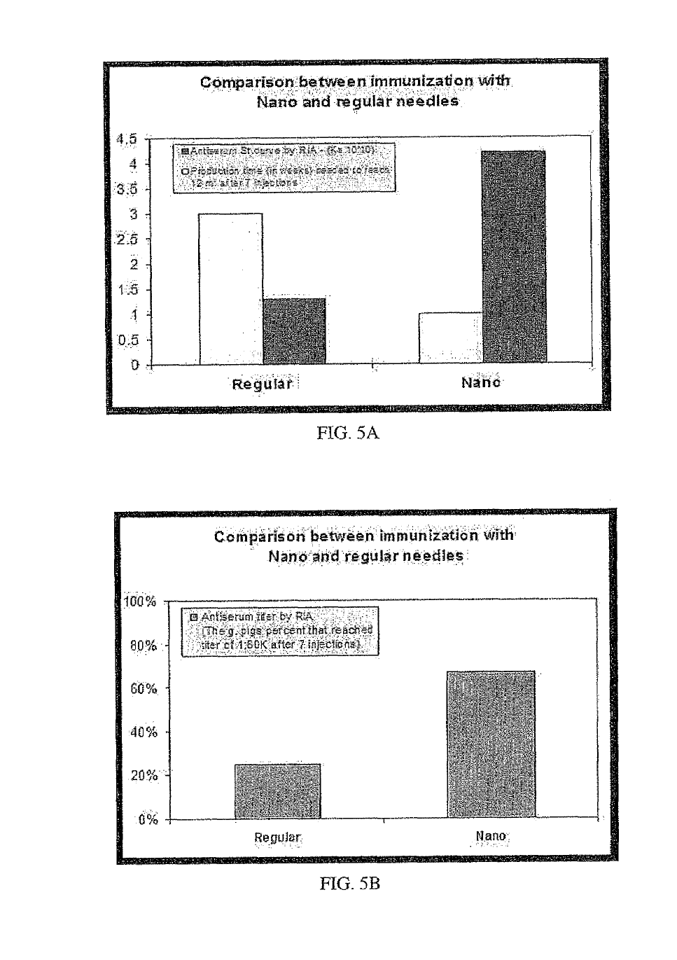

Turning now to the injection method of the present invention in more detail, FIGS. 2 and 3 each contain a sequence of numbered illustrations helpful for understanding the procedure used for injection. The sequence of FIG. 2 is useful for understanding the overall procedure while the side views of FIG. 3 show more clearly the geometrical relation between the device and the skin at each stage of the procedure. Each illustration will be referred to herein by the Figure number followed by the number of the illustration in parentheses. After an initial stage of disinfecting the region of skin to be injected (FIG. 2(1)), the microneedle device is pressed against the skin to cause the microneedles to penetrate into the skin of the subject, optionally holding the skin in place with a finger as shown in FIG. 2(2). It has been found particularly effective to perform the initial penetration with the syringe at a relatively steep angle, such as the 60 degree angle illustrated in FIG. 3(1). The device is then brought to an anchored state in which the microneedle is anchored in the skin and projects from the microneedle device in a direction having a major component parallel to the initial plane of the surface of the skin. This typically includes applying a force to the device in a direction having a component parallel to the skin surface, as shown in FIG. 2(3) and 2(4), achieving the “side insertion” state described above and illustrated in FIG. 4A where the microneedles are anchored into unstretched tissue not overlaid by the device. At this stage, effective anchoring of the microneedles may be visually verified by manipulating the device, as illustrated in FIG. 3(2).

The user preferably changes his or her grip on the syringe to remove her fingers from beneath the syringe, and the body of the syringe is lowered towards the skin surface, preferably reaching a position substantially flat against the skin as shown in FIGS. 2(5) and 3(3). The resulting pivotal motion preferably corresponds to angular motion through an angle of between about 5 degrees and about 30 degrees. As illustrated in FIG. 4B, this rotation has the effect of lifting the portion of the skin 100 in which the microneedles 12 are engaged above the initial plane of the surface of the skin, represented by dashed line 102. This lifting effect has been found to achieve enhanced shallowness of delivery while also enhancing sealing around the microneedles between the skin and the surrounding surfaces of the device. Contact pressure with the skin is preferably enhanced by gentle downward pressure applied by fingers of the user against the syringe, as illustrated in FIGS. 2(5) and 3(3). In the particularly preferred, but non limiting, option illustrated here, one finger applying pressure close to the microneedle interface itself helps to ensure sufficient downward pressure directly against the skin while a second finger maintains the position of the syringe substantially flat against the skin to maintain the lifting effect. While the portion of the skin is so lifted, a quantity of a biologically active agent is delivered intradermally via the microneedles by advancing the plunger of the syringe, as shown in FIG. 3(4). Successful completion of the injection is visibly verifiable through immediate formation of a distinctive white bleb from the very shallow intradermal delivery of the biologically active agent. The microneedles are then gently withdrawn from the skin as illustrated in FIGS. 2(6) and 3(5). The bleb disappears over a few minutes after completion of the injection as the fluid disperses in the adjacent tissue.

Accordingly, the method of the present invention may be used to intradermally deliver various agents selected from the group consisting of drugs, vaccines, diagnostic and therapeutic agents used in the prevention, diagnosis, alleviation, treatment, or cure of diseases to expose the injected agent to the microcirculatory blood vasculature and the lymphatic plexuses where the agent can be taken up, absorbed or can interact with the cells to obtain systemic delivery or an immune response in cases where the substance is either a drug or one or more vaccines. In this way, by targeting the dermal space, the immune response, pharmacokinetics (PK) and pharmacodynamics (PD) parameters of the agent can be dramatically altered, which, for example, in the case of improved bioavailability or improved immune response, can result in a reduction in the amount of the necessary dose of the substance to be delivered.

The agent may include a combination of drugs or vaccines. The drugs may include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as hirulog, IgE suppressors, Insulin, insulinotropin and analogs, Insulin-like growth factors, Interferons, Interleukins, Leutenizing hormone, Leutenizing hormone releasing hormone and analogs, Low molecular weight heparin, M-CSF, metoclopramide, Midazolam, Monoclonal antibodies, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, said vaccines, with or without carriers/adjuvants, include prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimers, H. Pylori, salmonella, diabetes, cancer, herpes simplex, human papilloma and like other agents include all of the major therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, antidepressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, sexual hypofunction and tranquilizers and major diagnostics such as tuberculin and other hypersensitivity and allergy agents.

The agent may also comprise a vaccine, with or without carriers, adjuvants and vehicles, said vaccine selected from the group consisting of prophylactic and therapeutic antigens including but not limited to subunit proteins, peptides, polypeptides, and polysaccharides, polysaccharide conjugates, toxoids, genetic based vaccines, live attenuated bacteria or viruses, mutated bacteria or viruses, reassortant bacteria or viruses; live, non-attenuated virus or viral vectors, inactivated or killed virus, live, non-attenuated bacteria and inactivated or killed bacteria or viruses, whole cells or components thereof, cellular vaccines or components thereof, viral and bacterial vectors including but not limited to those derived from adenoviruses, retroviruses alphaviruses, flaviviruses, and vaccinia viruses, vaccines in connection with addiction, anthrax, arthritis, cholera, diphtheria, dengue, tetanus, lupus, multiple sclerosis, parasitic diseases including Leishmania, psoriasis, Lyme disease, SARS, Ebola, Yellow Fever, meningococcus, measles, mumps, rubella, varicella, yellow fever, Respiratory syncytial virus, tick borne Japanese encephalitis, pneumococcus, smallpox, streptococcus, staphylococcus, typhoid, influenza including seasonal, pre-pandemic and pandemic influenza, hepatitis, including hepatitis A, B, C, D, E and G, otitis media, rabies, polio, HIV, parainfluenza, rotavirus, Epstein Barr Virus, CMV, chlamydia, non-typeable haemophilus, haemophilus influenza B (HIB), moraxella catarrhalis, human papilloma virus, tuberculosis including BCG, gonorrhoeae, asthma, atherosclerosis, malaria, E. coli, Alzheimer's Disease, H. pylori, salmonella, diabetes, cancer, herpes simplex, human papilloma, Yersitiia pestis, traveler's diseases, West Nile encephalitis, Carnplobacter, C. difficile and bioterrorism agents.

In a preferred embodiment, the agent is selected from a combination of vaccines against (i) measles, mumps and rubella, (ii) diphtheria, tetanus and acellular pertussis, (iii) hepatitis A and hepatitis B, (iv) haemophilus influenza B, diphtheria, tetanus and acellular pertussis, (v) haemophilus influenza B, hepatitis B, diphtheria, tetanus and a cellular pertussis, and (vi) haemophilus influenza B, inactivated polio, diphtheria, tetanus and acellular pertussis which are commonly delivered in combination to the subcutaneous and/or intramuscular space.

The invention encompasses methods of delivering the biologically active agents to the ID compartment so that the amount of the pre-selected dose of the agent deposited in the target tissue is increased compared to when the agent is delivered outside of the intradermal space, e.g., subcutaneous compartment (SC), intramuscular compartment (IM). Directly targeting the intradermal compartment as taught by the invention provides more rapid onset of effects of biologically active agents and higher bioavailability including tissue bioavailability, relative to other modes of delivery of such agents, including subcutaneous delivery. In addition, the invention suggests comparable responses to regular needle delivery for the ID mantoux application, the improvement in Mantoux due to more reliable and consistent delivery that does not penetrate too deep and thus does not provide “false negative” skin diagnostics. Agents delivered in accordance with the methods of the invention can be rapidly absorbed and systemically distributed via controlled ID administration that selectively accesses the dermal vascular and lymphatic microcapillaries, thus the agents may exert their beneficial effects more rapidly than SC administration.

### EXAMPLE 1

### Antibodies Production (Improved Immunogenicity)

The feasibility of the present method was proven effective in animal tests. Intradermal delivery using the microneedle device of the invention improves antibodies production. This example demonstrates the immunological benefits and the improvement in the kinetic profile of antibody production, using microneedle device and method of the present invention, in comparison to a regular ID delivery using a hypodermic needle.

### Description

Various animals (e.g., Guinea pigs) are used in industry to produce primary and secondary antibodies for biological and clinical research, based on immunoassay methods (e.g. ELISA). In some of these animals the specific antigen is injected intradermally, in order to achieve a larger and/or faster immune response (antibodies production). After the primary immunization, several immunization boosters are given at different time intervals, and blood is collected. The antibodies production level is measured by measuring the antigen/antibody complex formation levels, in different dilution levels, using conventional immunoassay methods. Usually, a fixed cut-off value (percentage of antigen/antibodies complex formation in a specific dilution level) must be achieved.

### Materials and Methods

18 Dunkin Hartley male Guinea Pigs were divided into two groups. The animals were anesthetized with a 0.2 ml of Ketamine/Xylazine mixture (1:1 ratio) I.M. Anesthetizing was followed by 0.1 ml I.D. immunization with Insulin from Porcine Pancreas (SIGMA, Cat. No. 1-5523) in a Freund's adjuvant emulsion mixture (SIGMA), injected in 4 sites on the skin. One group was immunized using a regular hypodermic 25 g needle, while the other group was immunized using the microneedle device of the invention. The microneedle device and injection procedure used in this and the other examples were according to the above description with reference to FIGS. 1A-4B, employing microneedles of height approximately 450 microns. At specific time points, the animals in both of the groups were given booster immunizations of the same amount as in the first immunization (total of 7 immunizations). Bleeding was performed by cardiac puncturing of the anesthetized animals, and withdrawing 4 ml of blood. After plasma separation, antigen/antibody complex levels were detected in the 2 ml serum sample, using a Dextran coated Charocal Radioimmunoassay method.

### Results and Discussion

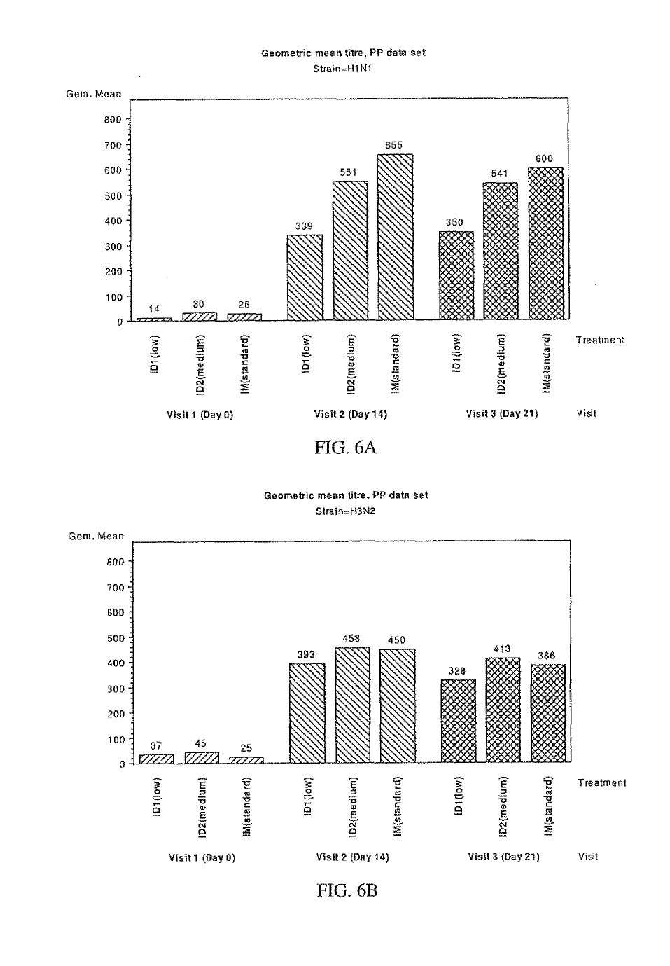

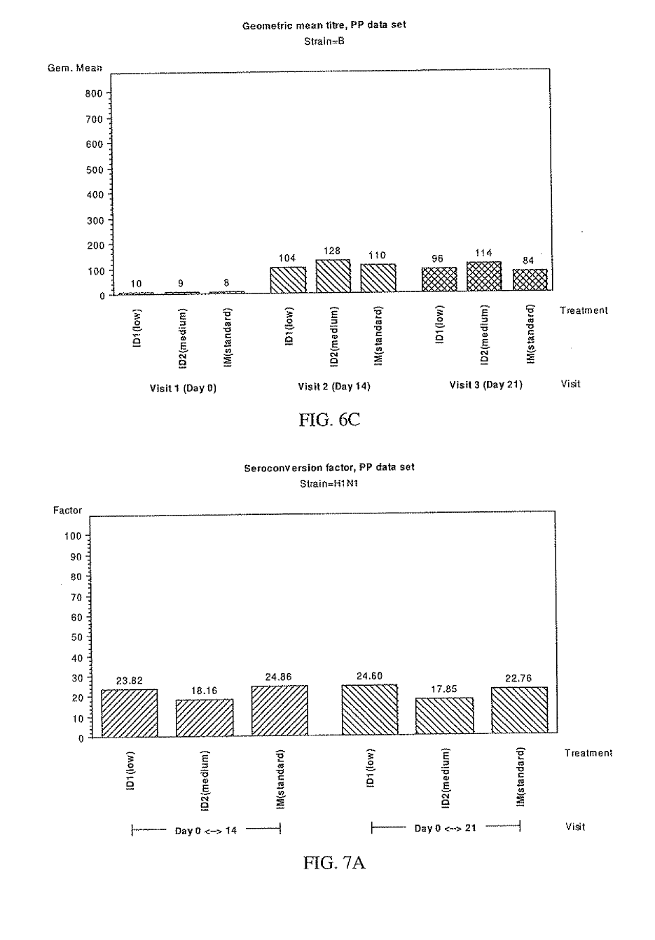

As can be seen from FIGS. 5A and 5B, the use of microneedles (marked as “Nano”) improved the immunological response—higher values of Antigen/Antibody complex formation were achieved at the dilution level of 1:80K (more than 3 times higher Kₐ [L/mol] values) in comparison to the regular injections using the regular 25 g hypodermic needle (marked as “Regular”).

Also, the production time needed to achieve the per-protocol cut-off value (50% Antigen/Antibody complex formation at a dilution level of 1:80 K) was 3 times shorter, which indicates a better kinetics.

### EXAMPLE 2

### Immunological Benefits: Clinical Trial

A pilot clinical trial was conducted in order to evaluate the immunogenicity of intradermal injections of low-dose flu vaccines delivered with the microneedle device of the invention (“MicronJet”). The standard dose delivered intramuscularly and the CPMP criteria for the annual re-licensure of influenza vaccines (9) were used as a reference for the above objective. Commercially available flu vaccine (Fluzone® (Sanofi Pasteur) winter 2006-2007) was used for all injections.

### Study Type:

Single center, single blinded, controlled study with three parallel groups.

### Study Population:

The study was conducted with healthy adult subjects. A total of 180 volunteers were recruited.

### Materials and Methods:

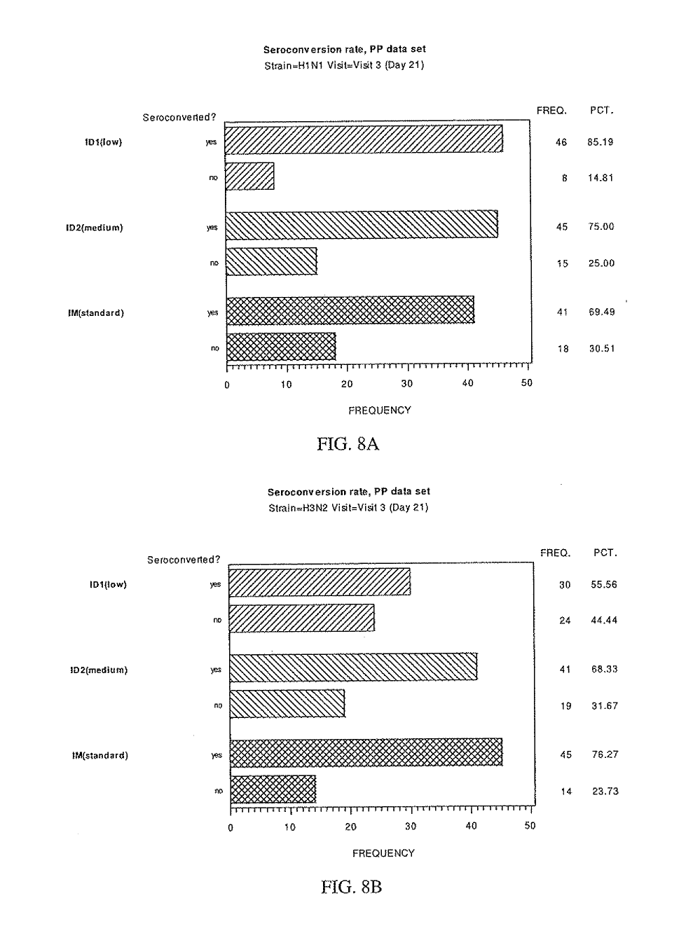

The study contained 3 arms. The sample size for each study arm was 60 subjects. This sample size is designed to meet annual licensing requirements for influenza vaccine in the EU. In the following analysis and in FIGS. 7-25, the following abbreviations are used for the three treatment arms:
ID1: low dose (20%) intradermal injection with the microneedle device of the invention (“MicronJet”). Subjects in this group received an intradermal injection of 20% of the standard IM vaccine dose (0.1 ml containing at least 3 μg of hemagglutinin antigen per strain).
ID2: medium dose (40%) intradermal injection with the microneedle device of the invention (“MicronJet”). Subjects in this group received an intradermal injection of 40% of the standard IM vaccine dose (0.2 ml containing at least 6 μg of hemagglutinin antigen per strain).
IM: standard dose intramuscular injection (control group). Subjects in this group received an intramuscular injection of the standard IM vaccine dose (0.5 ml containing at least 15 μg of hemagglutinin antigen per strain).

The vaccine used for all study arms was an approved, commercially available influenza vaccine (winter 2006-07) that was not altered in terms of concentration, components or formulation. Subjects were randomly assigned to each group.

A blood sample was retrieved from every subject upon entering the study and at 14 and 21 days post immunization in order to measure antibody titers and assess seroconversion and seroprotection.

The CPMP criteria for the annual re-licensure of influenza vaccines were used as a reference for the study objective. Immunogenicity parameters that were analyzed were: seroconversion, seroprotection, and GMT (geometric mean titer) fold increase.
Seroconversion: a significant immune response to the vaccine—guidelines refer to >40% of the subjects.
Seroprotection: a high immunity to the disease after the vaccination (this includes both subjects that responded well to the vaccine and subjects who were originally protected because they had the disease)—guidelines refer to >70% of the subjects.
GMT Fold Increase: a quantitative measurement of the immune response to the vaccine—the average value should be >2.5.

The three criteria were achieved in all study arms, as described below. CPMP guidelines were met in all tested strains, dosages, and in all methods of administration (ID, IM). No significant differences from IM were demonstrated. The results of this pilot study demonstrate that low dose intradermal administration of influenza vaccine using the microneedle device of the invention is sufficient to induce a protective immunological response against influenza in humans, based on accepted indicators and not significantly different from that of full dose IM delivery.

Specifically, influenza strain specific antibody titers were measured prior to the vaccination and at 14 and 21 days after the injection using a Hemagglutinin Inhibition test. Based on the antibody titers, seroconversion and seroprotection were determined. Antibody titers following vaccination as well as seroprotection rates represent an isolated state following vaccination and should be reviewed in light of baseline antibody titers (which differed significantly between the study groups). Seroconversion, however, accounts for baseline titers and thus provides a more accurate representation of the effect of vaccination.

### Strain Specific Antibody Titers

Antibody titers were measured against each of the three influenza strains represented in the vaccine (anti-H1N1, anti-H3N2 and anti-B antibodies) at the time points mentioned above.

The magnitude of the antibody response per treatment group was described as the geometric mean titer (GMT) for every given time point. The intensity of the immune response to the three influenza strains represented in the vaccine differed markedly, as demonstrated by differences up to a factor of 6 in the elicited GMTs.

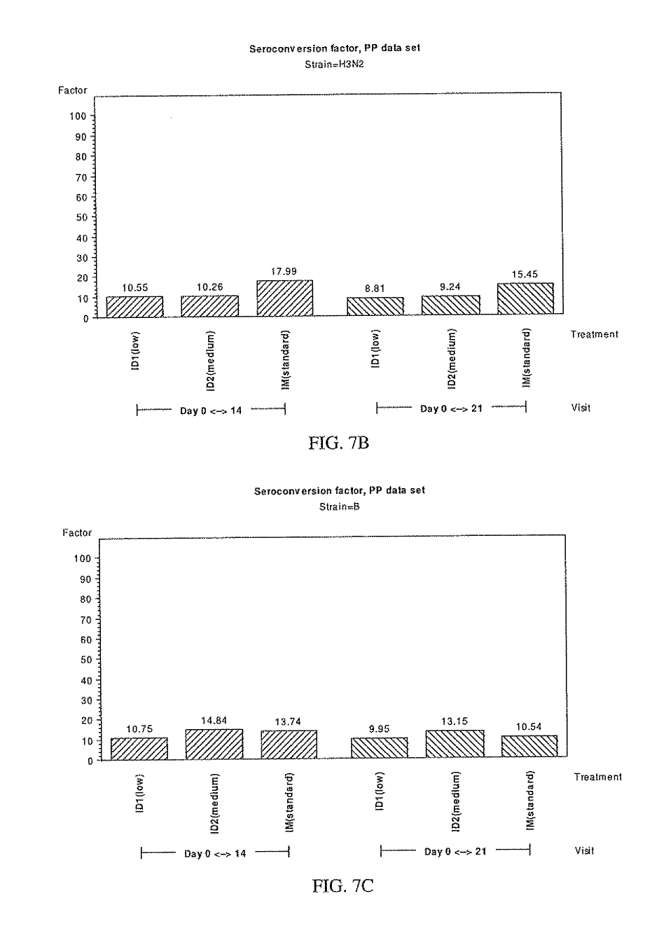

As shown in FIG. 6A, in the PP (per protocol) data set, the highest GMTs were observed against strain H1N1. In the IM group, a GMT of 655 was measured at Visit 2 (day 14) and a GMT of 600 at Visit 3 (day 21). Similar high GMTs were reached in the ID2 group (551 at Visit 2 and 541 at Visit 3). Notably lower GMTs were observed in the ID1 group (339 at Visit 2 and 350 at Visit 3), but baseline pre-vaccination titers were also visibly lower in this group as compared to the ID2 and IM groups.

As shown in FIGS. 6B and 6C, against the strains H3N2 and B slight differences in the GMTs between treatment groups were observed. However there were no markedly GMT differences between ID1 and ID2. As seen in FIG. 6B, the GMTs against H3N2 ranged between 393 and 458 at Visit 2 and between 328 and 413 at Visit 3.

As demonstrated in FIG. 6C, the lowest overall GMTs were elicited against strain B ranging between 104 and 128 at Visit 2 and 84 and 114 at Visit 3, with no marked differences between the study groups.

### Seroconversion

Seroconversion corresponds to a significant rise in antibody titer following vaccination, i.e., a 4-fold increase of the antibody titer or a post-vaccination titer of >1:40 for subjects with no predisposing antibodies before vaccination (a negative baseline titer). In the case of baseline titers below the limit of detection (marked as <10) an arbitrary value of 5 was used for the determination of seroconversion.

### Seroconversion Rates

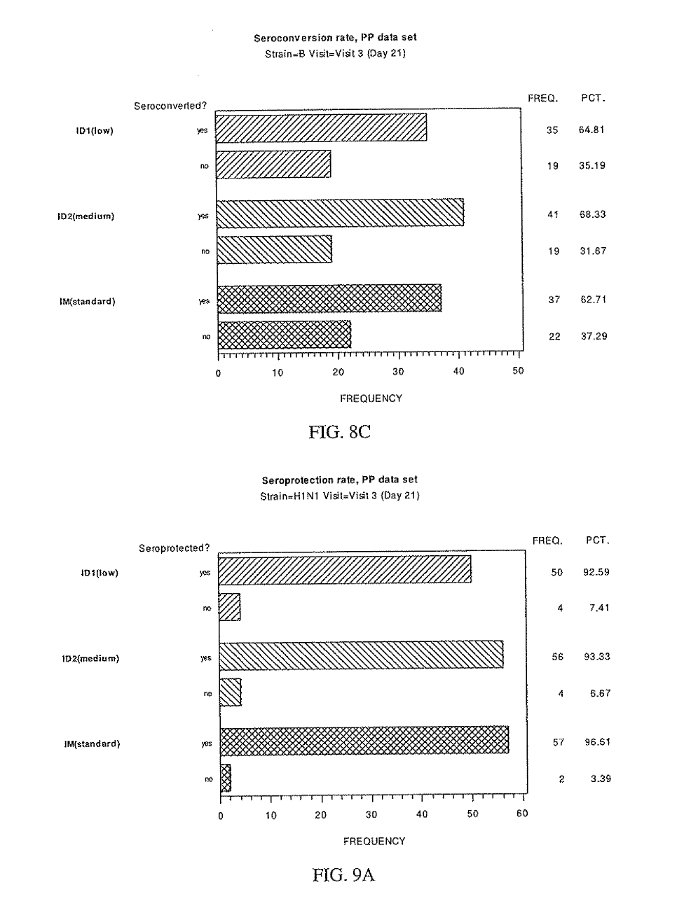

The seroconversion rates, i.e. the percentage of subjects who seroconverted at 14 days or 21 days after vaccination, are shown in FIGS. 8A-8C, where “FREQ” indicates frequency (number of subjects) and “PCT” indicates percentage.

As shown in FIG. 8A, in the PP data set, the highest seroconversion rate on Day 21 after the vaccination was achieved after MicronJet injections (85.19%) in the ID1 group against strain H1N1. In comparison, the seroconversion rate for the same viral strain in the ID2 group was 75.00% and in the IM group 69.49%.

As can be seen in FIG. 5B and FIG. 8C, seroconversion rates against strains H3N2 and B on Day 21 after vaccination were overall lower, compared to H1N1 for all study arms. As shown in FIG. 8B, seroconversion rates against strain H3N2 ranged from 55.56% (ID1 group) to 76.27% (IM group). The seroconversion rate for the ID2 group was 68.33%. As shown in FIG. 5C, seroconversion rates against strain B on Day 21 after vaccination were 62.71% in the IM group, 64.81% in the ID1 group and 68.33% in the ID2 group.

Seroconversion rates in subjects who received an intramuscular injection of the standard dose were overall not superior to the seroconversion rates in subjects who received low-dose ID injections with the MicronJet.

A comparison with the recommended seroconversion rate of 40% according to CPMP guidelines showed that all seroconversion rates determined for each influenza strain and treatment group at 14 and 21 days after the vaccination were statistically significantly higher than 40%. A statistical significance is defined by a p-value below 0.05. P-values testing if the seroconversion rate was significantly higher than 40% ranged from <0.0001 to max. 0.0098 in the PP dataset.

### Seroconversion Factors

In contrast to the seroconversion rate, the seroconversion factor provides quantitative information about the actual increase in antibody titers following vaccination. The seroconversion factor was defined as the quotient of the GMT at Day 14 or 21 and the GMT at Day 0.

Accordingly, the seroconversion factor was strongly dependent on baseline GMT values, which significantly differed between the treatment groups, especially against the strains H1N1 and H3N2. Different pre-existing immunities against influenza might depend on previous exposure or vaccination(s) against influenza.

As shown in FIG. 6A, the GMT baseline values against H1N1 in the PP data set differed markedly between ID1 and the ID2 and N group. Therefore even though absolute GMTs against H1N1 at Day 14 and 21 were lowest in the ID1 group (GMT 339 at Visit 2 and GMT 350 at Visit 3), the relative GMT increase in the ID1 group was not notably less or even higher (increase by a factor of 23.82 at Day 14 and 24.60 at Day 21), as shown in FIG. 7A. In the subjects of the ID2 group, who had a baseline GMT of 30, the GMTs only increased by a factor of 18.16 at Visit 2 and 17.85 at Visit 3, even though the absolute GMTs were significantly higher (GMT 551 at Visit 2 and GMT 541 at Visit 3) than in the ID1 group.

As shown in FIGS. 7B and 7C, the seroconversion factors for the strains H3N2 and B in the three treatment groups were overall slightly lower than for strain H1N1. As shown in FIG. 6B, baseline GMTs for H3N2 were 37 in the ID1 group, 45 in the ID2 group and 25 in the IM group. This explains why in the IM group the highest seroconversion factor was observed despite the fact that the highest GMT for Visit 2 and 3 was observed for the ID2 group.

Of note is that the seroconversion factors for strain B were not markedly less than for strain H3N2, even though overall low absolute GMT values were measured against strain B, as shown in FIG. 6C. (The absolute GMTs ranged from 104 to 128 at Visit 2 and 84 and 114 at Visit 3.) This was due to low baseline GMT values of 8 in the IM group, 9 in the ID2 and 10 in the ID1 group.

In all study groups, seroconversion factors for both Visit 2 and Visit 3 were notably higher than the 2.5 minimum required by the CPMP guideline, with p-values below 0.05.

### Seroprotection

Seroprotection was defined as a post vaccination titer of 1:40 or more. For all influenza strains the seroprotection rates in the subjects who received ID MicronJet injections were overall similar to the seroprotection rate in subjects who received an intramuscular injection. Seroprotection rates were higher than 80% for all study groups at both Day 14 and Day 21.

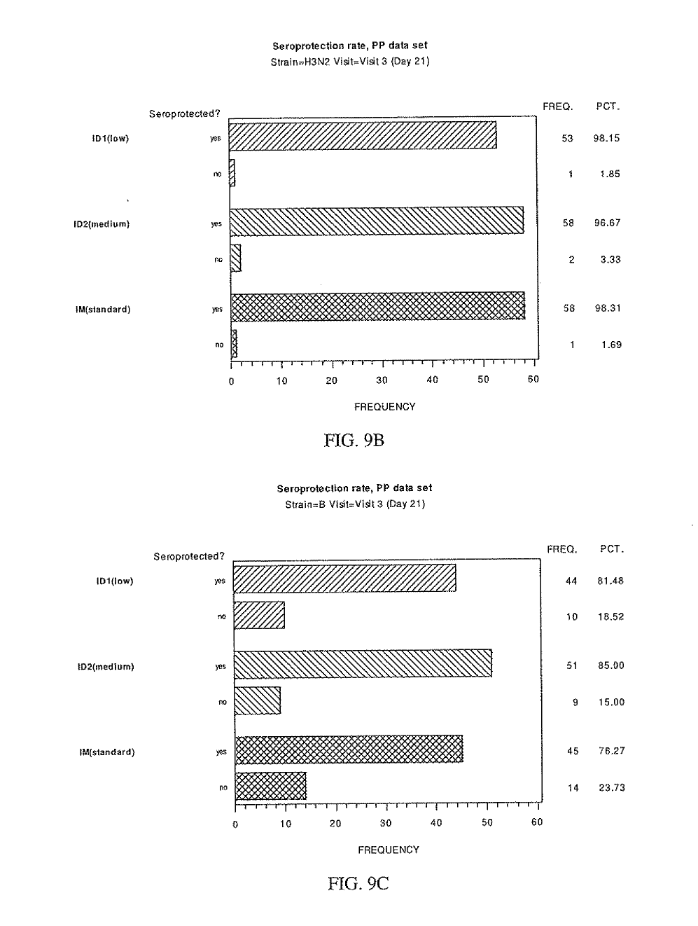

As shown in FIG. 9B, in the PP dataset, the highest seroprotection rates on Day 21 after vaccination were observed against strain H3N2, with 98.15% (ID1 group), 96.67% (ID2 group) and 98.31% (IM group) of the subjects being protected against strain H3N2. As shown in FIG. 9A, slightly fewer subjects were seroprotected against H1N1 on Day 21 after vaccination; the seroprotection rates were 92.59% in the ID1 group, 93.33% in the ID2 group and 96.61% in the IM group. As shown in FIG. 9C, the lowest seroprotection rates were observed against strain B. Slightly more subjects in the MicronJet groups (81.48% in the ID1 group and 85% in the ID2 group) were protected against strain B compared to subjects in the IM group (76.27%). Seroprotection rates on Day 14 after vaccination did not recognizably differ from seroprotection rates on Day 21 after vaccination.

Seroprotection rates were compared with a fixed reference rate of 70% that is recommended according to CPMP guidelines. Apart from one exception, all seroprotection rates measured in this study were statistically significantly higher than 70%, as demonstrated by p-values below 0.05. As shown in FIG. 9C, in the IM group, a seroprotection rate against the influenza strain B of 76.27% was observed at Visit 3. The corresponding p-value was 0.1466 indicating that the seroprotection rate was not significantly higher than 70% for this group.

It will be appreciated that the above descriptions are intended only to serve as examples, and that many other embodiments are possible within the scope of the present invention as defined in the appended claims.

### REFERENCES CITED

1. Laurent A et. al. Echographic measurement of skin thickness in adults by high frequency ultrasound to assess the appropriate microneedle length for intradermal delivery of vaccines. Vaccine 25 (2007) 6423-6430.
2. TUBERSOL® data sheet, Aventis Pasteur, Sep. 2001
3. P M Flynn, J L Shenep, L Mao, K Crawford, B F Williams and B G Williams, Influence of needle gauge in Mantoux skin testing. Chest 106:1463-5, 1994.
4. Halperin W, Weiss W I, Altman R, et al. A comparison of the intradermal and subcutaneous routes of influenza vaccination with A/New Jersey/76 (swine flu) and A/Victoria/75: report of a study and review of the literature. Am J Public Health 1979; 69: 1247-50.
5. Brown H, Kasel J A, Freeman D M, Moise L D, Grose N P, Couch R B. The immunizing effect of influenza A/NewJersey/76 (Hsw1N1) virus vaccine administered intradermally and intramuscularly to adults. J Infect Dis 1977; 136:Suppl:S466-S471.
6. Kenney, Richard T., Frech, Sarah A., Muenz, Larry R., Villar, Christina P., Glenn, Gregory M. Dose Sparing with Intradermal Injection of Influenza Vaccine. N Engl J Med 2004 351; 2295-2301.
7. Belshe R B, Newman F K, Cannon I, et al. Serum antibody responses after intradermal vaccination against influenza. N Engl J Med 2004; 351:2286-2294.
8. Safety and immunogenicity of varying dosages of trivalent inactivated influenza vaccine administered by needle-free jet injectors; Vaccine. 2001 Sep. 14; 19(32):4703-9
9. CPMP note for guidance on harmonization of requirements for influenza vaccines. London: The European Agency for the Evaluation of Medicinal Products, 1997:1-18. (CPMP/BWP/214/96).

What is claimed is:

1. A method of intradermally delivering at least one biologically active agent into the skin of a subject, comprising the steps of:
(a) providing a microneedle device including at least one hollow microneedle;
(b) causing the at least one microneedle to penetrate into the skin of the subject and assume an anchored state in which the microneedle is anchored in the skin and projects from the microneedle device in a direction having a major component parallel to the initial plane of the surface of the skin;
(c) performing a pivotal motion of the microneedle device such that the portion of the skin in which the at least one microneedle is engaged is lifted above the initial plane of the surface of the skin; and
(d) while the portion of the skin is lifted, delivering intradermally via the microneedle a quantity of a biologically active agent.

2. The method of claim 1, wherein said pivotal motion is performed such that a region of the device behind the microneedle is pressed against the skin, thereby enhancing sealing against the skin.

3. The method of claim 1, wherein said pivotal motion is performed by angular motion through an angle of between about 5 degrees and about 30 degrees.

4. The method of claim 1, wherein the microneedle device is associated with a syringe, and wherein said pivotal motion is performed by pressing the syringe substantially flat against the surface of the skin.

5. The method of claim 1, wherein said microneedle device comprises:
(a) a skin contact surface which is brought into substantially facing relation with the skin surface in said anchored state;
(b) a front surface angled relative to the skin contact surface and meeting the skin contact surface at an edge, the at least one hollow microneedle being deployed so as to project from the front surface adjacent to said edge; and
(c) a rear surface angled relative to the skin contact surface and meeting the skin contact surface at a pivot region about which said pivotal motion is performed.

6. The method of claim 5, wherein said at least one hollow microneedle is implemented as a plurality of hollow microneedles deployed in a line adjacent to said edge.

7. The method of claim 1, wherein said at least one microneedle is formed on a substrate, said microneedle projecting from said substrate to a height of between about 250 and about 750 microns.

8. The method of claim 1, wherein said agent is selected from the group consisting of drugs, vaccines, peptides, proteins, carbohydrates, nucleic acid molecules, lipids, organic molecules, biologically active inorganic molecules, and combinations thereof used in the prevention, diagnosis, alleviation, treatment, or cure of diseases.

9. The method of claim 8, wherein said nucleic acid molecules include DNA, cDNA, RNA, siRNA, oligonucleotides, genes and a fragment thereof.

10. The method of claim 8, wherein said drugs include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as hirulog, IgE suppressors, Insulin, insulinotropin and analogs, Insulin-like growth factors, Interferons, Interleukins, Leutenizing hormone, Leutenizing hormone releasing hormone and analogs, Low molecular weight heparin, M-CSF, metoclopramide, Midazolam, Monoclonal antibodies, Narcotic analgesics, nicotine, Non-steroid anti-inflamatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, said vaccines, with or without carriers/adjuvants, include prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and B, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimers, H. Pylori, salmonella, diabetes, cancer, herpes simplex, human papilloma and like other agents include all of the major therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, anti-depressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, sexual hypofunction and tranquilizers and major diagnostics such as tuberculin and other hypersensitivity and allergy agents.

11. The method of claim 8, wherein said agent is a vaccine, with or without carriers, adjuvants and vehicles, said vaccine selected from the group consisting of prophylactic and therapeutic antigens including but not limited to subunit proteins, peptides, polypeptides, and polysaccharides, polysaccharide conjugates, toxoids, genetic based vaccines, live attenuated bacteria or viruses, mutated bacteria or viruses, reassortant bacteria or viruses; live, non-attenuated virus or viral vectors, inactivated or killed virus, live, non-attenuated bacteria and inactivated or killed bacteria or viruses, whole cells or components thereof, cellular vaccines or components thereof, viral and bacterial vectors including but not limited to those derived from adenoviruses, retroviruses alphaviruses, flaviviruses, and vaccinia viruses, vaccines in connection with addiction, anthrax, arthritis, cholera, diphtheria, dengue, tetanus, lupus, multiple sclerosis, parasitic diseases including Leishmania, psoriasis, Lyme disease, SARS, Ebola, Yellow Fever, meningococcus, measles, mumps, rubella, varicella, yellow fever, Respiratory syncytial virus, tick borne Japanese encephalitis, pneumococcus, smallpox, streptococcus, staphylococcus, typhoid, influenza including seasonal, pre-pandemic and pandemic influenza, hepatitis, including hepatitis A, B, C, D, E and G, otitis media, rabies, polio, HIV, parainfluenza, rotavirus, Epstein Barr Virus, CMV, chlamydia, non-typeable haemophilus, haemophilus influenza B (HIB), moraxella catarrhalis, human papilloma virus, tuberculosis including BCG, gonorrhoeae, asthma, atherosclerosis, malaria, E. coli, Alzheimer's Disease, H. Pylori, salmonella, diabetes, cancer, herpes simplex, human papilloma, Yersitiia pestis, traveler's diseases, West Nile encephalitis, Carnplobacter, and C. difficile and bioterrorism agents.

12. The method of claim 8, wherein said agent is selected from a combination of vaccines against (i) measles, mumps and rubella, (ii) diphtheria, tetanus and acellular pertussis, (iii) hepatitis A and hepatitis B (iv) haemophilus influenza B, diphtheria, tetanus and acellular pertussis, (v) haemophilus influenza B, hepatitis B, diphtheria, tetanus and a cellular pertussis, and (vi) haemophilus influenza B, inactivated polio, diphtheria, tetanus and acellular pertussis which are commonly delivered in combination to the subcutaneous and/or intramuscular space.

13. The method of claim 1, wherein the agent is at least one vaccine and wherein a comparable immune response is induced using a lesser dose of the agent as compared to when the same agent is delivered via an intramuscular route.

14. The method of claim 1, wherein said agent achieves a better response using the same dose, or an equivalent response using a lesser dose, lesser adjuvant or no adjuvant, faster onset, longer response, improved memory response, higher bioavailability, lesser boosts, and lesser toxicity compared to when the same agent is delivered via an intramuscular route.

15. The method of claim 1, wherein the agent achieves a similar response in less time or a greater response in similar time as compared to when the same agent is delivered via an intramuscular route.

16. The method of claim 1, wherein said agent achieves a greater antibody production yield by generating higher titers for the same period of time or reduction of boosts required for the target titer or faster arrival to target titers or a combination thereof as compared to when the same agent is delivered via an intramuscular or subcutaneous route.

17. The method of claim 1, wherein said agent is delivered at a controlled rate, volume, pressure and depth to generate a Mantoux skin response more reliably and consistently than the traditional intradermal Mantoux method with reduced false negative skin diagnostics.
